(19) United States
(12) Patent Application Publication
(10) Pub. No.: US 2018/0255834 A1
(43) Pub. Date: Sep. 13, 2018

(54) A METHOD AND A DISPENSING ARRANGEMENT FOR TREATING A FLOW OF VAPORIZED LIQUID PROVIDED BY AN E-CIGARETTE AND AN E-CIGARETTE FOR THE ORAL INTAKE OF VAPORIZED LIQUID CONTAINING AN ADDITIONAL COMPOSITION

(71) Applicant: Alexandra DILLMANN, Meckenbeuren (DE)

(72) Inventors: Alexandra DILLMANN, Mackenbeuren (DE); Katharina BIZU, Wiggensbach (DE)

(73) Assignee: AD BEAUTY GMBH, Meckenbeuren (DE)

(21) Appl. No.: 15/760,139

(22) PCT Filed: Sep. 16, 2016
(86) PCT No.: PCT/EP2016/072032

§ 371 Date: Mar. 14, 2018

(30) Foreign Application Priority Data
Sep. 16, 2015 (EP) 15185550.9
Sep. 16, 2015 (EP) 15185555.8

(51) Int. Cl. A24F 47/00; A24B 15/16; A24B 15/30; A24B 15/34; A24B 15/32; A24B 15/38; A24B 15/42; A24B 15/40; A61K 31/07; A61K 31/51; A61K 31/375; A61K 31/355; A61K 31/593; A61K 31/455; A61K 31/519; A61K 31/4188; A61K 31/198; A61K 33/04; A61K 33/30; A61K 31/525; A61K 31/4415; A61K 31/714; A61K 31/205; A61K 31/197; A61K 33/06; A61K 31/202; A61K 31/122; A61K 31/05; A61K 38/39; A61K 36/185; A61K 36/8945; A61K 31/5685; A61K 31/728; A61K 31/661; A61K 33/00; A61K 31/133; A61K 36/16; A61K 36/324; A61K 36/87; A61K 36/889; A61K 36/77; A61K 31/522; A61M 11/04; A61M 15/06

(52) U.S. Cl. CPC A24F 47/008; A61M 2205/3653; A24B 15/303; A24B 15/345; A24B 15/30; A24B 15/32; A24B 15/38; A24B 15/42; A24B 15/302; A24B 15/301; A24B 15/308; A24B 15/403; A24B 15/406; A24B 15/385; A61K 31/07; A61K 31/51; A61K 31/375; A61K 31/355; A61K 31/593; A61K 31/455; A61K 31/519; A61K 31/4188; A61K 31/198; A61K 33/04; A61K 33/30; A61K 31/525; A61K 31/4415; A61K 31/714; A61K 31/205; A61K 31/197; A61K 33/06; A61K 31/202; A61K 31/122; A61K 31/05; A61K 38/39; A61K 36/185; A61K 36/8945; A61K 31/5685; A61K 31/728; A61K 31/661; A61K 33/00; A61K 31/133; A61K 36/16; A61K 36/324; A61K 36/87; A61K 36/889; A61K 36/77; A61K 31/522; A61M 11/042; A61M 15/06; A61M 2205/8206; A24B 15/167

(57) ABSTRACT

The present invention relates to a dispensing arrangement (1), or to an e-cigarette (100) containing such a dispensing arrangement, for treating a flow of vaporized liquid provided via a flow channel (3) of an e-cigarette, comprising a dispensing unit (10) for adding an additional composition into the flow of vaporized liquid of the e-cigarette, and a composition reservoir (2) for retaining the additional composition, wherein the dispensing unit is in communication with a composition reservoir and in communication with the flow channel, wherein the composition reservoir is arranged separately to the flow channel, and wherein the dispensing arrangement is adapted to temper the vaporized liquid and/or the additional composition to a temperature of below 40° C. or below 35° C. before adding the additional composition into the flow of vaporized liquid, as well as methods for the oral intake of compositions, particularly those comprising temperature-sensitive compounds or compounds insoluble in the liquid carrier, such as certain anti-aging compounds, by virtue of an e-cigarette comprising said dispensing arrangement. The present invention also relates to uses of said dispensing arrangement or of an e-cigarette comprising said dispensing arrangement for the oral intake of additional compositions comprising at least one anti-aging compound to a user.

### TECHNICAL FIELD

The present invention relates to a dispensing arrangement, or to an e-cigarette containing such a dispensing arrangement, for treating a flow of vaporized liquid provided via a flow channel of an e-cigarette, as well as methods for the oral intake of compositions, particularly those comprising temperature-sensitive compounds or compounds insoluble in the liquid carrier, such as certain anti-aging compounds, by virtue of an e-cigarette comprising said dispensing arrangement. The present invention also relates to uses of said dispensing arrangement or of an e-cigarette comprising said dispensing arrangement for the oral intake of additional compositions comprising at least one anti-aging compound to a user.

### TECHNOLOGICAL BACKGROUND

There are about one billion smokers in the world. During the combustion of a conventional cigarette, a tar mist containing nicotine is generated, which is the major “active ingredient” of a cigarette. When inhaling the smoke, the tar mist containing nicotine enters pulmonary alveoli and is quickly absorbed to the smoker's blood releasing the active ingredient as well as other, often hazardous by-products, such as cyanide, benzene, formaldehyde, carbon monoxide, and nitrogen oxide.

In view of the negative health issues associated with conventional smoking, some substitute cigarettes, such as an electronic cigarette have been developed in recent years. They typically include a mouthpiece, a battery, a liquid chamber and an atomizer. Such an electronic cigarette further contains a liquid, which may contain nicotine and optionally also other components such as flavorants. An example for the addition of flavor substances is e.g. disclosed in Chinese patent CN 101926506 B.

An electronic cigarette may also include liquids that are devoid of nicotine and other tobacco-derived products, allowing the user/consumer to enjoy the pleasure from the act of smoking without, however, inhaling nicotine, tar and other potentially hazardous substances.

The electronic cigarette typically works by interaction with the user/consumer where the user/consumer sucks on a mouthpiece of the electronic smoking device. By creating slight underpressure (partial vacuum), the liquid in the liquid chamber flows into the nebulizing or vaporizing chamber for nebulization or vaporization (sometimes also referred to as atomization) of the liquid, yielding an aerosol that leaves the device through the mouthpiece, thereby simulating the function of a conventional cigarette. The temperature at which the atomization takes place is typically between 65° and 120° C., which is typically achieved by a heating element driven by an energy source such as an internal battery in the vaporizing device. An example for an e-cigarette is e.g. disclosed in WO 2012062247 A1.

It is, however, known that many compounds which can possibly be used as additives in an e-cigarette, or similar devices such as electronic pipes or water pipes (“e-shishas”) are not stable at such temperatures, or at least undergo serious degradation, thus limiting the selection of possible additives in practice.

The trend for a healthier lifestyle continues unabated and has led to an increased awareness among consumers that the natural diet, especially these days where a lot of food is highly processed, does not provide an optimal mix of nutrients, vitamins and other compounds known to be beneficial for one's health and well-being. Accordingly, many consumers supplement their regular food intake with supplements, typically in the form of tablets or liquid solutions or suspensions. These include vitamins, dietary fibers and other extracts known to exert beneficial effects on the health and dietary status of the consumer.

Among the supplements regularly taken by consumers is a group of substances that are known (or suspected) to exert an “anti-aging” effect, i.e. an effect that slows down or halts (and ideally reverts) the biological process of cell aging. However, in order to have a beneficial effect on consumers' health status, most of these compounds should be taken regularly, which requires a high degree of self-discipline.

Having regard to the situation as discussed above, it is therefore one object of the invention to provide new and advantageous modes of administration of anti-aging compounds, which are capable of offering good consumer acceptance and help ensure compliance with the recommended regular intake schemes for such compounds.

### SUMMARY OF THE INVENTION

Considering the above, it is an object of the present invention to provide a dispensing arrangement for an e-cigarette with an improved ability for the intake of additional compounds, particularly of temperature-sensitive compounds such as certain anti-aging compounds. In the present context, the term e-cigarette is not intended to be limiting to e-cigarettes, but shall refer to all types of electric or electronic cigarettes, electric or electronic pipes and electric or electronic water pipes, respectively.

Accordingly, in a first aspect of the present invention, a dispensing arrangement for treating a flow of vaporized liquid provided via a flow channel of an e-cigarette is provided, which comprises

(i) a dispensing unit for adding an additional composition into the flow of vaporized liquid of the e-cigarette; and
a composition reservoir for retaining the additional composition,
wherein the dispensing unit is in communication with the composition reservoir and in communication with the flow channel, and further
wherein the composition reservoir is arranged separately to the flow channel;
characterized in that
the dispensing arrangement is adapted to temper the vaporized liquid and/or the additional composition to a temperature of below 40° C. or below 35° C. or below 30° C., or to room temperature (i.e. ambient temperature) before adding the additional composition into the flow of vaporized liquid.

Preferably, the mixture of the additional composition and the vaporized liquid that is intended to enter the oral cavity of the consumer (also referred to herein as “user”) has a temperature of below 40° C., or even below 35° C., or below 30° C. or room temperature. A consumer in the present context includes, in general, any mammal, though in most cases the consumer will of course be a human subject.

By providing the above-described dispensing arrangement, it is possible to add an additional composition from the dispensing unit into the flow of vaporized liquid without the need of heating and vaporizing it, because the addition of the composition takes place subsequently to, or separately from, vaporizing the liquid. Accordingly, this dispensing arrangement allows temperature-sensitive compounds, compounds having a poor solubility in the liquid to be vaporized, and/or compounds which may be harmful to or negatively interact with the components for vaporizing the liquid to be vaporized to be added into the vaporized liquid. A user's body can thus be provided with components and agents (such as anti-aging compounds) which were not exposed to physical, chemical and/or physico-chemical destruction or decomposition prior to entering the oral cavity of the user.

The composition reservoir should generally be capable of reliably retaining the additional composition. Furthermore, by adjusting the reservoir's fill capacity, a certain amount of the additional composition, e.g. a daily dose, can be provided. To guarantee an unhindered flow of the vaporized liquid in the flow channel, the composition reservoir is arranged spatially separated from the flow channel.

Due to the communication of the dispensing unit with the composition reservoir and the flow channel, a secure and steady dosage of the amount of the additional composition which is added into the flow of vaporized liquid can be provided for each intake.

In certain embodiments, the composition reservoir is replaceable and/or refillable. Accordingly, a very efficient and ecological usage of the dispensing arrangement can be provided. Instead of exchanging the whole dispensing arrangement when the competition reservoir is empty or its filling level is low, preferably below a certain level, merely the composition reservoir needs to be replaced or refilled.

The composition reservoir of the dispensing arrangement comprises in certain embodiments a porous solid body coated, and/or impregnated, and/or soaked with the additional composition. In these embodiments, the dispensing unit may be provided in the form of passageways in the porous solid body.

In other embodiments, the composition reservoir is or comprises a container for receiving the composition in the form of a liquid, powder, or other solid form. Accordingly, by retaining the additional composition in a container, the additional composition can be reliably retained within the dispensing arrangement of an e-cigarette. Thus, a leakage or spilling of the additional composition and hence an unwanted staining of other parts of the dispensing arrangement and/or the e-cigarette can be avoided. Thus, a robust dispensing arrangement having a simple design can be provided. The composition reservoir merely has to be placed in a reservoir seating. Since the dispensing unit is formed by the passageways, no further components are necessary for adding the additional composition into the flow of vaporized liquid.

Furthermore, in preferred embodiments when the composition reservoir is located radially outwards to the flow channel, the composition reservoir can be replaced or refilled with minimal effort since it is easy to reach by the user/consumer.

In some embodiments, the dispensing arrangement according to the present invention further comprises a cooling device for cooling the additional composition and/or the flow channel such that the additional composition has a temperature of lower than 40° C. or lower than 35° C. and/or the vaporized liquid has a temperature of lower than 40° C. or lower than 35° C., or lower than 30° C. In some embodiments, the additional composition and/or the vaporized liquid thus may have room or ambient temperature, or may even be cooled to a temperature of below 15° C., or below 10° C. or below 3° C. In certain embodiments, the cooling device is preferably provided in the form of a cooling surface or cooling fins at the outer shell of the dispensing device and/or the e-cigarette.

In particular for providing a temperature below room temperature, such as below 10° C., or below 3° C., the dispensing arrangement may in some embodiments comprise at least one Peltier element for cooling. The at least one Peltier element is preferably supplied with electric energy by a power supply of the e-cigarette. Thus, the dispensing arrangement according to the present invention can be used even for highly temperature-sensitive compounds. Furthermore, functioning of the dispensing arrangement can be maintained even when exposed to sunlight and/or a heating caused by external heat sources, such as solar radiation.

This arrangement allows supplying the body of a user with compounds contained in the additional composition directly and with fill effect, without exposing them to possibly devastating effects caused by contacting the additional compounds with a heating element typically used to vaporize the liquid generated in an e-cigarette. In other words, temperature-sensitive compounds, or compounds having a poor solubility in the liquid to be vaporized, or compounds which may be harmful to the components for vaporizing the liquid to be vaporized can be added into the vapor. Accordingly, a user's body can be provided with components and agents which were not previously exposed to physical, chemical and/or physico-chemical destruction or decomposition.

Furthermore, it combines the positive consumer experience associated with the inhalation of a vapor or smoke, i.e. a solid/gaseous aerosol, from an e-cigarette acting as a pleasure-giving stimulant with the intake of compounds known for their positive effect on preventing aging effects, thereby increasing consumer compliancy and regularity of administration.

In some embodiments, the dispensing unit comprises at least a valve, nozzle, capillary, wick or passageway and/or opening.

The additional composition preferably enters the oral cavity of the consumer in the form of a vapor, an aerosol, a (liquid/liquid) emulsion or a (liquid/solid) suspension, wherein the droplets, gas particles, or solid particles preferably are respirable.

In the context of the present invention, an aerosol may include fine solids or small drops of liquid compounds in the air or gas phase. A vapor usually denotes a substance in gaseous form, typically with some condensation. In the latter case, the vapor is essentially similar to a liquid/gaseous aerosol.

The dispensing arrangement includes in some embodiments at least a connector for connecting the dispensing arrangement and the e-cigarette, wherein said connector is arranged at a side facing the e-cigarette. In some embodiments, the dispensing arrangement includes, alternatively or in addition, a mouthpiece or a mouthpiece connection which is arranged at a side facing the user.

Preferably, the connector is provided in form of a standard connector, like a plug or a socked such as a drip tip connector, or a thread, preferably a so called 510-thread or an eGo-thread, such that the dispensing arrangement is attachable to common available e-cigarettes. Furthermore, by the mouthpiece connection, which is likewise provided as a standard connection, common available mouthpieces can be attached to the dispensing arrangement. Hence, the dispensing arrangement can be used with ordinary e-cigarette-equipment. When the dispensing arrangement also comprises the mouthpiece, an ordinary mouthpiece merely has to be replaced by the dispensing arrangement to provide a fully functional e-cigarette.

The dispensing arrangement comprises in some embodiments at least one air inlet opening such that a fluidic connection is provided through the composition reservoir from the at least one air inlet opening to the dispensing unit. Hence, when applying a negative pressure to the flow channel, an additional air flow into the flow channel is provided since the additional air is sucked in through the air inlet openings, flows through the composition reservoir and enters the flow channel via the dispensing unit. The additional composition may thereby be added into the additional air flow. This additional air flow is then mixed with the flow of vaporized liquid inside the flow channel and guided into the user's throat. Due to the additional air flow having ambient temperature, an additional cooling of the vaporized liquid is provided, allowing a very compact design of the e-cigarette. Furthermore, by the same token, a uniform distribution of the additional composition in the flow of vaporized liquid can be achieved.

In some embodiments, the additional air flow is mixed with the flow of vaporized liquid after leaving the mouthpiece, preferably through a separate opening of the mouthpiece for the additional air flow. Thus, the additional air flow is mixed with the flow of vaporized liquid inside the user's mouth and/or throat.

The composition reservoir of the dispensing arrangement according to the present invention may in some embodiments further include an additional composition. This additional composition may in preferably contain at least one temperature-sensitive compound, and/or at least one compound having a poor solubility in the vaporized liquid, and/or at least one anti-aging compound. In some embodiments, the additional composition included in the composition reservoir will preferably comprise at least one temperature-sensitive anti-aging compound.

In another aspect, the present invention relates to an e-cigarette for the oral intake of a vaporized liquid, comprising a liquid reservoir for retaining a liquid, a flow channel and a vaporizing unit for vaporizing the liquid, wherein the e-cigarette further includes a dispensing arrangement as described herein, i.e. with a dispensing unit for adding an additional composition into the flow of vaporized liquid of the e-cigarette.

The dispensing arrangement of said e-cigarette is in some embodiments connectable with the e-cigarette by means of a counterpart connector. Alternatively, the dispensing arrangement may also be integrated in the e-cigarette.

In some embodiments, the dispensing unit is spaced apart from a vaporizing chamber of the vaporizing unit, such that a cooling section is provided in the flow channel between a heater element for vaporizing the liquid located in the vaporizing chamber and the dispensing unit, wherein in the cooling section, the vaporized liquid is able to cool down. Preferably, the cooling section is provided in a length such that the vaporized liquid is able to cool down to a temperature of below 40° C. or below 35° C., or below 30° C.

For controlling the vaporizing unit and thus the vaporization of the liquid, a control circuit arrangement may optionally be provided, which is preferably controllable by a switch. The control circuit arrangement may optionally comprise a processor element. Furthermore, a power supply, preferably in form of a battery, more preferably a rechargeable battery, may be arranged in the e-cigarette for supplying the vaporizing unit and/or the control circuit arrangement with electric energy.

In certain embodiments, a sensor arrangement comprising at least one sensor is provided for measuring the temperature of the vaporized liquid and/or the temperature of the additional composition and/or the temperature of the heater element and/or the pressure in the flow channel and/or the vaporizing chamber and/or the filling level of the composition reservoir and/or the liquid reservoir and/or the status of the power supply. Preferably, the sensor arrangement is in communication with the control circuit arrangement.

In some embodiments, at least a supply air inlet is arranged in fluidic connection with the vaporizing chamber for providing a supply air flow supporting the flow of the vaporized liquid. Preferably, the at least one supply air inlet comprises a closure which opens when a negative pressure is applied to the vaporizing chamber or when a switch for operating the e-cigarette is pressed. Alternatively, the closure is controlled by the control circuit arrangement. The flow of vaporized liquid hence is a combination of vaporized liquid and the supply air.

In certain embodiments, a negative pressure (i.e. an “underpressure”/low pressure below atmospheric pressure) can be applied to the flow channel of said e-cigarette, thereby allowing the addition of the additional composition into the flow of vaporized liquid by a passive intake mechanism due to the negative pressure. Preferably, the negative pressure may be generated by the user/consumer through suction at a mouthpiece outlet of the e-cigarette or the dispensing arrangement. Alternatively, a unit capable of producing a negative pressure, i.e. lowering the pressure inside the flow channel below ambient pressure, may be provided.

Alternatively or in addition, the addition of the additional composition in the e-cigarette is achieved by a controlled injection of the additional composition into the flow of vaporized liquid.

Another aspect of the present invention relates to a method for treating a flow of vaporized liquid provided by an e-cigarette, wherein said method comprises the steps of:
providing a liquid to be vaporized;
providing an additional composition;
vaporizing the liquid;
causing a flow of the vaporized liquid, and
adding the additional composition into the flow of vaporized liquid;
   wherein, before adding the additional composition into the flow of vaporized liquid, the temperature of the vaporized liquid and/or the additional composition is/are tempered to a temperature below 40° C., or below 35° C., or below 30° C. or room temperature; and/or
   wherein the mixture of the additional composition and the vaporized liquid has a temperature of below 40° C. or below 35° C., or below 30° C. or room temperature.

In some embodiments of this aspect of the invention, the additional composition comprises at least one temperature-sensitive compound, and/or at least one compound having a poor solubility in the vaporized liquid, and/or at least one anti-aging compound. Preferably, the additional composition comprises at least one temperature-sensitive anti-aging compound.

In certain embodiments of this method, the additional composition is directly added into the flow of vaporized liquid. Alternatively, the additional composition is added into an additional air flow which is then mixed with the flow of vaporized liquid.

The step of adding the additional composition into the flow of vaporized liquid is in some embodiments achieved by applying a negative pressure which causes a passive intake of the additional composition into the flow of vaporized liquid. Alternatively or in addition, the step of adding the additional composition into the flow of vaporized liquid is achieved by injecting the additional composition into the flow of vaporized liquid in a controlled fashion.

Yet another aspect of the invention relates to the use of a dispensing arrangement or of an e-cigarette comprising said dispensing arrangement as defined herein for the oral intake of an additional composition comprising at least one anti-aging compound.

The composition reservoir comprises the additional composition containing at least one anti-aging compound preferably in solid or in liquid form. In some embodiments, the additional composition comprising the at least one anti-aging compound is present in the form of a vapor, an aerosol, an emulsion or a suspension.

Thus, in some embodiments of this aspect of the invention, the vaporization of the composition comprising at least one anti-aging compound and, optionally, the oral intake is accomplished by generating a partial vacuum (i.e. lower than ambient pressure) within the reservoir comprising the additional composition within the e-cigarette, thereby converting the liquid or solid composition comprising said at least one anti-aging compound into a vapor or aerosol, which can for example be inhaled by the consumer.

The partial vacuum may be generated by the e-cigarette itself by any suitable means (manual, electrically powered), or it may be generated by the user of said device, for example by suction on the mouthpiece of said e-cigarette. The lower pressure serves to or aids in vaporizing or nebulizing the composition (and, optionally the “liquid” generating the regular vapor in said e-cigarette), and may further be used to withdraw the vapor, aerosol, emulsion or suspension from the e-cigarette.

The oral intake of the composition comprising the at least one anti-aging compound may in some embodiments also take place—entirely or partially—as a homogenous liquid solution, as a heterogeneous emulsion (heterogeneous liquid/liquid), or as a suspension (heterogeneous solid/liquid mixture).

In certain embodiments, the composition comprising at least one anti-aging compound is converted to a vapor, an aerosol, an emulsion or a suspension prior to the oral intake at a temperature of below about 40° C., preferably below about 35° C., or below about 30° C., or even at room/ambient temperature.

In some embodiments of this aspect of the present invention, the composition reservoir comprises a vaporizable or nebulizable liquid composition comprising a liquid carrier composition and the at least one anti-aging compound. Preferably, the vaporizable liquid composition is in such embodiments present in the form of a solution, emulsion or dispersion.

The liquid carrier composition may in some embodiments comprise a mixture of at least one liquid polyalcohol and water. For example, the liquid carrier may comprise a mixture of 1,2-propanediol and water, which may optionally further comprise 1,2,3-propanetriol (i.e. glycerol) and/or ethanol.

Suitable compounds or compositions having a known anti-aging effect in the context of the present invention include plant extracts or natural compounds isolated from plants, certain amino acids, peptides, or proteins, certain fatty acids and their derivatives, certain hormones and hormone-active substances, certain vitamins and co-factors, organic silica and a number of metals (metal ions).

An anti-aging effect is generally understood to refer to slowing down or reversing the processes of aging in order to extend both the maximum and average lifespan. However, in the context of the present invention, an “anti-aging compound” also includes compounds known to have a positive “anti-age” effect on skin, hair, nails, and to improve the function of the subject's immune and nervous systems.

Anti-aging compounds according to the present invention can be roughly divided in terms of their function/effect in the body of the consumer. Thus, anti-aging compounds according to the present invention include, but are not limited to

i) compounds having a beneficial effect on the function of the immune system
ii) compounds having a beneficial effect on the function of the nervous system, such as improving stress resistance
iii) compounds having a beneficial effect on hairs, nails, skin, body shape
iv) compounds having a beneficial effect on male and female libido, maintaining normal spermatogenesis and fertility, and increased peripheral blood flow.

Alternatively, anti-aging compounds according to the present invention may be classified according to their chemical nature and mode of action. Examples include anti-oxidants, essential vitamins and co-factors required for enzymatic reactions involved in anti-aging processes, and hormone or hormone-like compounds, etc.

In general, any compound for which a beneficial anti-aging effect has been established can be employed in the context of the present invention. The anti-aging compounds should preferably be of pharmaceutical grade (USP).

The person of skill in the art will generally know, or can find out with a few routine experiments, how to bring the anti-aging compounds and compositions comprising them in a form that can be used with a vaporizing device such as an e-cigarette, e-pipe, or e-shisha.

Those of skill in the art will furthermore be aware that some anti-aging compounds are heat-sensitive, for example proteins, or certain vitamins. In such cases, the compositions comprising such heat-sensitive anti-aging compounds should ideally be convertible into a vapor/aerosol/emulsion/suspension at lower temperatures of below 40° C. or below 35° C. (e.g. by selecting an appropriate liquid carrier, by avoiding contact with the heating coil or wire, or by employing partial vacuum to achieve vaporization or nebulization at lower temperatures).

In the context of the present invention, the at least one anti-aging compound may preferably be selected from the group consisting of:

Acai-berry extract, Acerola extract, BCAA extract, Boswellia extract, Caviar extract, Cistus Incanus, Damiana leaf extract, Ginkgo-Biloba extract, Pomegranate seed extract, Green Coffee extract, OPC-grape seed extract, Prunus Cerasus/bitter cherry extract, Sabal fruits extract, Potency Wood extract, Wild Yams Root extract, Zingiber Officinals/ginger root extract, Butyrospermum Parkii (shea butter), Guarana powder, caffeine, L-theanine, collagen, collagen hydrolysate, creatine monohydrate, hyaluronic acid, L-carnitine, astaxanthin, bioflavonoids, dimethylaminoethanol (DMAE), dehydroepiandrosteron (DHEA), omega-3 fatty acids, docosahexaenic acid (DHA), eicosapentaenic acid (EPA), L-arginine, L-glutamine, L-isoleucine, L-leucine, L-lysine, L-valine, phosphonoserine, Somatotropin (HGH, Human Growth Hormone), Vitamin A (retinol), Vitamin B1 (thiamine), Vitamin B2 (riboflavin), Vitamin B3 (niacin), Vitamin B5 (pantothenic acid), Vitamin B6 (pyridoxine), Vitamin B7 (biotin), Vitamin B9 (folic acid), Vitamin B12 (cobalamin), Vitamin C (ascorbic acid), Vitamin D (ergo-/cholecalciferol), Vitamin E (tocopherol), alpha-tocotrienol, Vitamin K, Coenzyme Q10, organic silica, pycnogenol, resveratrol, magnesium, selenium, zinc, gold particles, and mixtures of any of the foregoing compounds or compositions/extracts.

Certain anti-aging compounds according to the present invention belong to the family of inorganic or organic acids. Further examples of anti-aging compounds include glycosaminoglycans such as hyaluronic acid, magnesium salts of citric acid, amides such as urea, biochemical messenger molecules such as hormones, mineral supplements, pure silver ions, various plant extracts, polysaccharides containing fructose such as inulin (e.g. from Cichorium intybus), chitin, polyphenolic fruit powders such as black currant powder (Ribes nigrum), acai berry powder (Euterpe oleracea), beet root powder (Beta vulgaris), soluble fibers such as cacao fibers (Theobroma cacao), aminosugars such as D-glucosaminesulfate-2-Potassium chloride (from crustaceans), sugar molecules such as rhamnose, oils such as Canola oil, olive oil or argan oil, proteins such as collagen (from fish), plant enzymes such as papain, bromelain or ficin, lipids such as ceramides (from water-insoluble natural compounds), peptides such as Matryxil, enzymes, various complexes of lipopeptides such as pentavitin, plant hormones such as phytohormones, plant or animal stem cells or algae.

In some embodiments, the composition comprising the at least one anti-aging compound contains at least 2, 3, 4, 5, 6, 7, 8, 9, or at least 10 different anti-aging compounds, which may be carefully selected and combined to achieve a desired effect on the skin, hair, nails, the immune and nervous systems, etc. Suitable compositions including a variety of anti-aging compounds according to the present invention are also described in the Example section below.

In certain embodiments where the composition contains a vitamin with a known anti-aging effect, the composition may contain at least one further anti-aging compound that is not a vitamin, for example from the list set out above. Such an anti-aging compound, which is not a vitamin thus includes but is not restricted to Zingiber Officinalis/Ginger Root Extract, Prunus Cerasus/Bitter Cherry Extract, Butyrospermum Parkii, Somatotropin (HGH, Human Growth Hormone), 5 Caviar extract, gold particles, and Alpha-Tocotrienol. In some embodiments, the anti-aging compound is selected from Zingiber Officinals/Ginger Root Extract, Prunus Cerasus/Bitter Cherry Extract, or Butyrospermum Parkii. In some embodiments, the anti-aging compound is Somatotropin (HGH, Human Growth Hormone). In some embodiments, the anti-aging compound is selected from caviar extract or gold particles. In other embodiments, the anti-aging compound is selected from Alpha-Tocotrienol.

In some embodiments, the composition may, besides the at least one anti-aging compound, further comprise one or more flavoring agents (flavorants), for example to increase consumer acceptance or to mask any unwanted odor of one of the other components in the composition. Examples of such flavorants are generally well-known and may include natural extracts, essential oils, natural or synthetic food additives and the like. Such flavorants may typically be added to the compositions in an amount of between 1 and 15% of the total weight of the liquid carrier composition, though the exact amount will obviously depend on the flavorant in question.

It will be understood that the upper limit for the additional composition is generally governed by the size and capacity of the dispensing arrangement or the e-cigarette, in particular its composition reservoir. Moreover, it is preferred that the maximum recommended daily dosage or recommended intake amount for a given anti-aging compound (which data is in almost all cases publically available) is not exceeded. Likewise, although there is no technical lower limit for the amount of anti-aging compound in the composition reservoir of the e-cigarette, it will of course be appreciated that below a certain concentration, no compound will exert any relevant anti-aging effect. In any event, identifying suitable amounts of the anti-aging compounds is considered to be routine for those of skill in the art. Likewise, whether an anti-aging composition can be included in the vaporizing device in a liquid or in solid form depends on the solubility of the chosen composition in the first or second liquid. Thus it will be apparent that certain low solubility compounds may preferably be included in solid form so that the compound will reach the oral cavity of the consumer as fine particles.

In summary, the present invention provides a novel mode of administration of additional compounds such as anti-aging compounds through an e-cigarette which provides a solution to the above-mentioned objects can be provided, thereby improving the compliance of the consumer with regard to the regular intake of anti-aging compounds, required to have a long-lasting beneficial effect on the consumer, and combining the pleasure derived from the act of “smoking”, i.e. the active inhalation of a vapor or aerosol, with the desire of the consumer to supplement the regular diet with compounds known for their beneficial effect on preventing unwanted aging processes. Thus, the positive consumer experience associated with the inhalation of a vapor, or smoke, i.e. a solid/gaseous aerosol, from an e-cigarette acting as a pleasure-giving stimulant with the intake of compounds known for their positive effect on preventing aging effects, thereby increasing consumer compliancy and regularity of administration.

Having described the various aspects of the present invention in general terms, it will be apparent to those of skill in the art that many modifications and slight variations are possible without departing from the spirit and scope of the present invention.

### BRIEF DESCRIPTION OF THE DRAWINGS

The present disclosure will be more readily appreciated by reference to the following detailed description when being considered in connection with the accompanying drawings in which:

FIG. 1 is a schematic sectional view of an e-cigarette having a dispensing arrangement;

FIG. 2 is a schematic sectional view of another exemplary embodiment of an e-cigarette comprising a separate dispensing arrangement;

FIG. 3 is a schematic perspective exploded view of a further embodiment of a vaporizing unit and an attached dispensing arrangement of an e-cigarette;

FIG. 4 is a schematic perspective view of the dispensing arrangement of FIG. 3;

FIG. 5 is a schematic perspective view of another embodiment of a dispensing arrangement; and

FIG. 6 is a schematic exploded view of the dispensing arrangement of FIG. 5.

### DETAILED DESCRIPTION OF PREFERRED EMBODIMENTS

In the following, the invention will be explained in more detail with reference to the accompanying Figures. In the Figures, like elements are denoted by identical reference numerals and repeated description thereof may be omitted in order to avoid redundancies.

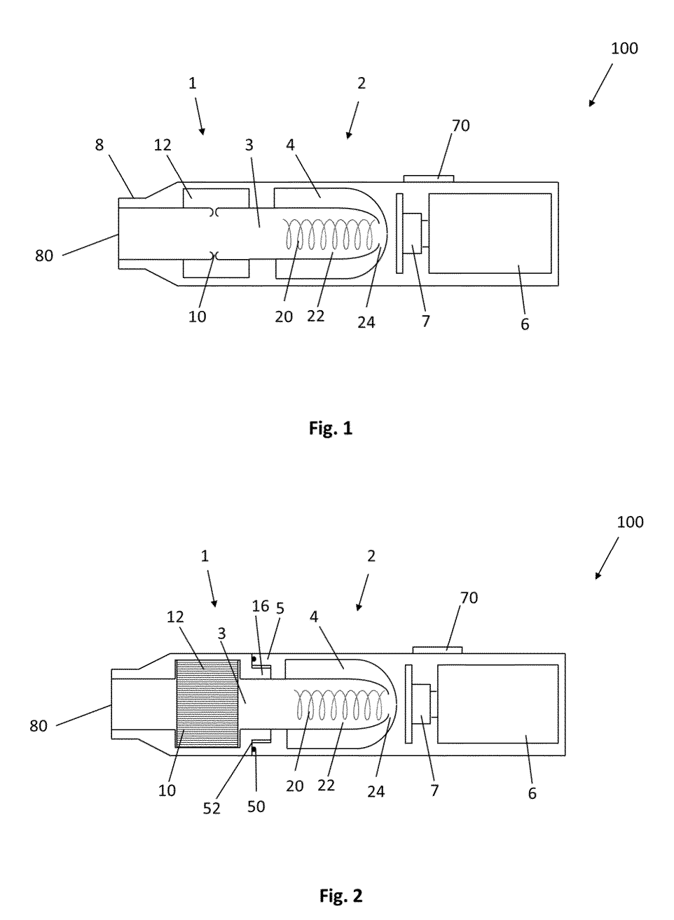

FIG. 1 shows a schematic sectional view of an e-cigarette 100 having a dispensing arrangement 1. In this embodiment, the dispensing arrangement 1 is incorporated into the e-cigarette 100. The e-cigarette 100 is further provided with a vaporizing unit 2 which comprises a liquid reservoir 4 for retaining a liquid to be vaporized and further a heater element 20 in form of a heating coil arranged in a vaporizing chamber 22, wherein the liquid reservoir 4 opens into the vaporizing chamber 22 via a reservoir outlet 24. A power supply 6 in form of a battery for supplying the vaporizing unit with electric energy is arranged adjacent the vaporizing unit 2. For controlling the vaporizing unit 2 and thus the vaporizing of the liquid, a control circuit arrangement 7 is provided controllable by a switch 70 which can be operated by a user. Furthermore, the vaporizing chamber 20 is connected to a flow channel 3 configured to guide the flow of the vaporized liquid to a mouthpiece outlet 80.

Between the vaporizing unit 2 and the mouthpiece outlet 80, a separate composition reservoir 12 retaining an additional liquid carrier composition comprising an anti-aging-compound to be added into a flow of vaporized liquid in form of a hollow cylinder is arranged around the flow channel 3, wherein a fluidic connection between the composition reservoir 12 and the flow channel 3 is provided by means of a dispensing unit 10. The dispensing unit 10 in this embodiment is provided in form of a nozzle. Alternatively, the dispensing unit 10 can also be provided in form of at least a perforation, valve or any other suitable orifice, or in form of at least a capillary or a wick.

When a user is sucking on the mouthpiece for operating the e-cigarette, a negative pressure is applied to the flow channel 3 and thus the vaporizing chamber 22. As a consequence, liquid is lead from the liquid reservoir 4 into the vaporizing chamber 20 by means of capillary action. When the switch 70 is operated by the user, the heater element 20 vaporizes the liquid at a temperature between 65° C. and 120° C. By the act of sucking, the vaporized liquid is caused to flow towards the mouthpiece opening 80. On its way from the vaporizing chamber 22 to the mouthpiece outlet 80, the flow of vaporized liquid passes the dispensing unit 10. Since the heater element 20 and the dispensing unit 10 are spaced apart from another, a cooling section is provided in the flow channel 3 between the heater element 20 and the dispensing unit 10, in which the vaporized liquid is able to cool down such that the vaporized liquid has a temperature below 35° C. at the dispensing unit 10. The adding of the composition into the flow of vaporized liquid is caused by a passive intake through the dispensing unit 10 due to the negative pressure in the flow channel 3. Thereby, the nozzle shape of the dispensing unit 10 causes a nebulization of the additional composition such that the additional composition is converted into an aerosol having a respirable droplet size, wherein the aerosol is evenly distributed in the flow of vaporized liquid when passing the mouthpiece outlet 80.

In addition, a unit capable of producing a negative pressure, i.e. lowering the pressure inside the flow channel 3 below ambient pressure, may be provided. Instead of a passive intake, the composition can also be actively injected by the dispensing unit 10 wherein the dispensing unit 10 then preferably is provided in form of an injection valve.

In FIG. 2 a schematic sectional view of another exemplary embodiment of an e-cigarette 100 comprising a separate dispensing arrangement 1 is shown. The dispensing arrangement 1 is connected with a vaporizing unit 2 according to the vaporizing unit shown in FIG. 1 via a connector 16 which is in communication with a counterpart connector 5 arranged at the vaporizing unit 2. Here, the connector 16 is provided with an external thread and the counterpart connector 5, consequently, is provided with an internal thread. For sealing the connection between the dispensing arrangement 1 and the vaporizing unit 2, a sealing 50 is provided at the end face 52 of the counterpart connector 5.

The dispensing arrangement 1, furthermore, is integrally connected to a mouthpiece 8 having a mouthpiece outlet 80. Between the mouthpiece outlet 80 and the vaporizing chamber 22, again, a flow channel 3 is provided for guiding the flow of vaporized liquid.

Inside the flow channel 3, a composition reservoir 12 in form of a porous solid body impregnated with an additional composition is arranged, wherein the composition reservoir is spaced apart from the vaporizing chamber 22. The porous solid body, in this embodiment, is a steel wool cylinder. Here, a dispensing unit 10 for adding the additional composition into the flow of vaporized liquid is provided in the form of passageways in the porous solid body. Hence, when the vaporized liquid flows through the passageways, droplets of the additional composition are dragged along the flow of vaporized liquid and thereby added into the flow of vaporized liquid.

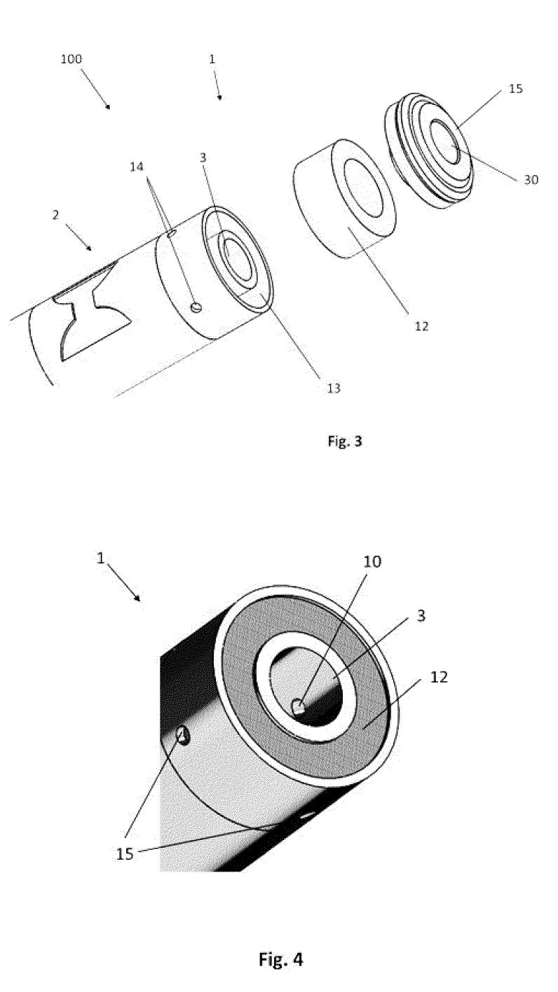

FIGS. 3 and 4 show a further embodiment of a vaporizing unit 2 and an attached dispensing arrangement 1 of an e-cigarette 100, wherein FIG. 3 shows a schematic perspective exploded view of the dispensing arrangement 1 of the e-cigarette 100 and FIG. 4 shows a schematic perspective view of the dispensing arrangement 1. In this exemplary embodiment, a composition reservoir 12 comprising an anti-aging-compound is provided in form of a hollow cylinder consisting of steel wool and is arranged in a reservoir seating 13 separate to the flow channel 3. The dispensing unit 10 in this embodiment is formed by passageways through the porous solid body and by openings connecting the flow channel 3 and the separate reservoir seating 13. By means of air inlet openings 14 provided on an outer shell 26 of the e-cigarette 100, a fluidic connection is provided through the reservoir seating 13 and hence through the composition reservoir 12 from the one air inlet openings 14 to the dispensing unit 10.

Further, a detachable cap 15 is provided for capping the reservoir seating 13 such that the composition reservoir 12 can easily be replaced or refilled with the additional composition. The cap 15 comprises a mouthpiece connection in form of a connection socket 30 for receiving a connector plug of a mouthpiece. Here, the connection socket 30 is adapted to receive a so called drip tip connector plug. Thus, commonly available mouthpieces can be connected to the e-cigarette 100. The connection socket 30 furthermore functions as an outlet of the flow channel 3.

When a user is sucking on the mouthpiece and thereby applying a negative pressure to the flow channel 3, ambient air is sucked through the air inlet openings 14, flowing through the reservoir seating 13 and the dispensing unit 10 into the flow channel 3. Hence, an additional air flow into the flow channel 3 is provided. Since the additional air sucked in through the air inlet openings 14 flows through the composition reservoir 12 seated in the reservoir seating 13, the additional composition is added into the additional air flow. When, at the same time, the vaporizing unit 2 is operated by the user, a flow of vaporized liquid is created likewise to those in FIGS. 1 and 2. The additional air flow comprising the additional composition and the flow of vaporized liquid are mixed in the flow channel 3 downstream the dispensing unit 10 such that the flow leaving a mouthpiece outlet into the user's throat is a mixture of vaporized liquid, ambient air and the additional composition. Furthermore, an additional cooling of the vaporized liquid is provided by the additional air flow having ambient temperature. Hence, the e-cigarette can have a very compact design.

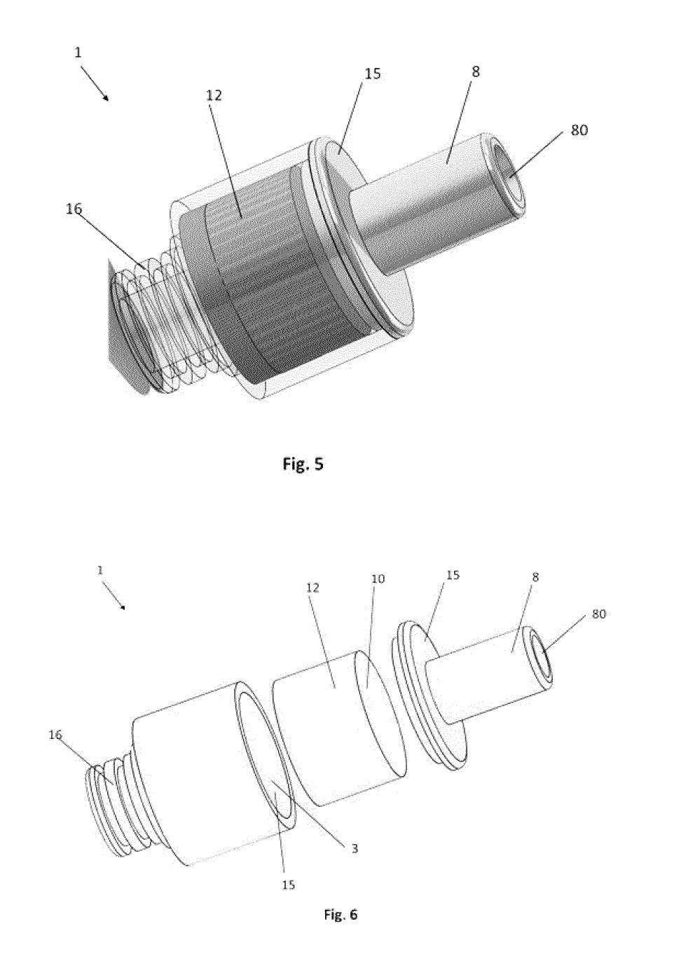

In FIG. 5, a schematic perspective view of another embodiment of a dispensing unit 1 is shown. FIG. 6 shows the dispensing arrangement 1 of FIG. 5 in a schematic exploded view. Likewise the dispensing unit depicted in FIG. 2, the dispensing unit 1 comprises a cylindrical composition reservoir 12 formed by steel wool, which is seated in a reservoir seating 13 inside the flow channel 3 extending from a connector 16 to a mouthpiece opening 80 of the dispensing arrangement 1. The dispensing unit 1 in this embodiment is formed as a mouthpiece 8. For this purpose, the connector 16 of the dispensing arrangement 1 is provided in form of a drip tip plug. Hence, the dispensing arrangement 1 can be connected to commonly available e-cigarettes, wherein an ordinary mouthpiece is replaced by the dispensing arrangement 1. For replacing or refilling the composition reservoir 12, the dispensing arrangement 1 comprises a detachable cap 15. According to the embodiment of FIG. 2, the additional composition is added into a flow of vaporized liquid emanating from a vaporizing unit to which the dispensing arrangement 1 is connected in that the vaporized liquid flows though passageways in the composition reservoir 12 which functions as dispensing unit 10.

It will be obvious for a person skilled in the art that these specific embodiments only depict examples of a plurality of possibilities. Hence, the embodiments described herein should not be understood to form a limitation of these features and configurations. Any possible combination and configuration of the described features can be chosen according to the scope of the invention.

The following examples describe a variety of compositions comprising at least one and in many cases multiple anti-aging compounds. These exemplary compositions are naturally understood to illustrate but not limit the invention in any way. Any of those compositions may be combined with or dissolved in a suitable liquid carrier, or can be used as is, in liquid or solid form, as the case may be.

### EXAMPLES

### Example 1: Compositions for Enhancing Immune System Function

Below are listed some illustrative composition comprising at least one, and in most cases several anti-aging compounds that can be employed in the context of the present invention.

### Composition 1:

| | | |
|---|---|---|
| | Recommended daily | Composition |
| Compound | intake | (Example) |

| | | | | |
|---|---|---|---|---|
| Vitamin A* (retinol) | 0.8-1.0 | mg | 0.5-1.5 | IU |
| Vitamin B1 (thiamine) | 1.0-1.2 | mg | 200 | μg |
| Vitamin C (ascorbic acid) | 100 | mg | 500 | μg |
| Vitamin E (Tocopherol) | 10 | mg | 300 | μg |

*3.33 IU Vitamin A = ~1 μg retinol = ~6 μg carotene

### Composition 2:

| | Recommended daily | Composition |
|---|---|---|
| Compound | intake | (Example) |
| Cholecalciferol (Vitamin D) | 20 μg (800 IU)* | 5 μg |

*Sum of skin production and food intake

### Composition 3:

| | | |
|---|---|---|
| | Recommended daily | Composition |
| Compound | intake | (Example) |

| | | | | |
|---|---|---|---|---|
| Vitamin B3 (niacin) | 15-20 | mg | 500 | μg |
| Vitamin B9 (folic acid) | 60-600 | μg | 48 | μg |
| Vitamin B7 (biotin) | 30-60 | μg | 10.5 | μg |

### Composition 4:

| | Recommended daily | Composition |
|---|---|---|
| Compound | intake | (Example) |
| L-Lysine | 3 g | 500 μg |

Further additives that may be combined with the above L-Lysine include the following:

| | Recommended daily | | Composition | |
|---|---|---|---|---|
| Compound | intake | | (Example) | |
| Selenium | 100 | μg | 10 | μg |
| Zinc | 30 | mg | 10 | μg |
| Bioflavonoids | 5.1 | mg | 100 | μg |
| Vitamin B2 (riboflavin) | 1.5-6 | mg | 20 | μg |
| Vitamin B6 (pyridoxine) | 1.6-6 | mg | 10 | μg |
| Vitamin B9 (folic acid) | 60-600 | μg | 150 | μg |
| Vitamin B12 (cobalamin) | 3-6 | μg | 10 | μg |
| Vitamin C (ascorbic acid) | 100-500 | mg | 80 | μg |
| Vitamin D (ergo-/cholecalciferol) | 114 | μg | 10 | μg |

### Example 2: Composition for Improving Skin, Hair and Body Shape

### Composition 5:

| | Recommended daily | Composition | |
|---|---|---|---|
| Compound | intake | (Example) | |
| L-Glutamine | 440.0 mg | 25 | μg |
| L-Carnitine | 320.0 mg | 2 | mg |
| L-Arginine | 160.0 mg | 1.5 | μg |

Further additives that may be combined with the above anti-aging compounds include the following:

| | Recommended daily | | Composition | |
|---|---|---|---|---|
| Compound | intake | | (Example) | |
| Vitamin B1 (thiamine) | 3.8 | mg | 3.5 | μg |
| Vitamin B2 (riboflavin) | 4.9 | mg | 4.6 | μg |
| Vitamin B3 (niacin) | 56.0 | mg | 500 | μg |
| Vitamin B5 (pantothenic acid) | 21.0 | mg | 21 | mg |
| Vitamin B6 (pyridoxine) | 4.9 | mg | 40 | μg |
| Vitamin B9 (folic acid) | 700 | μg | 7 | μg |
| Vitamin B7 (biotin) | 150 | μg | 1.5 | μg |
| Vitamin B12 (cobalamin) | 8.8 | μg | 0.1 | μg |
| Magnesium | 100.0 | mg | 330 | μg |
| Selenium | 90 | μg | 1 | μg |
| Zinc | 30 | mg | 3 | μg |

### Example 3 Composition for Improving Blood Flow and Male Libido

### Composition 6:

| | Recommended daily | Composition |
|---|---|---|
| Compound | intake | (Example) |
| L-Arginine | 3000 mg | 150 mg |

### Example 4 Composition for Lowering Cholesterol, Triglycerides

### Composition 7:

| | Recommended daily | Composition |
|---|---|---|
| Compound | intake | (Example) |
| Omega 3 fatty acids | 1400 mg | 1400 μg |

Further additives that may be combined with the above Omega 3 fatty acids include the following:

| | Recommended daily | | Composition |
|---|---|---|---|
| Compound | intake | | (Example) |
| Eicosapentaenic acid (EPA) | 800 | mg | 800 μg |
| Docosahexaenic acid (DHA) | 400 | mg | 400 μg |
| Vitamin E | 10 | mg | 100 μg |

### Example 5 Compositions with Coenzyme Q10 for Improving Skin Appearance and Reducing Wrinkles

### Composition 8:

| | Recommended daily | Composition |
|---|---|---|
| Compound | intake | (Example) |
| Coenzyme Q10 | 100 mg | 5-50 mg |
| (ubichinone - 10) | | |

### Example 6 Compositions for Improving Stress Resistance

### Composition 9:

| | Recommended daily | Composition |
|---|---|---|
| Compound | intake | (Example) |
| L-Carnitine | 1200 mg | 200-500 mg |

### Example 7 Compositions with Resveratrol

### Composition 10:

| | Recommended daily | Composition |
|---|---|---|
| Compound | intake | (Example) |
| Resveratrol | 1500-2500 mg | 1500-2000 μg |

Further additives that may be combined with the above Resveratrol include the following:

| | | |
|---|---|---|
| | Recommended daily | Composition |
| Compound | intake | (Example) |

| | | | | |
|---|---|---|---|---|
| Co-Q10 | 100 | mg | 1000 | μg |
| Vitamin C | 1000 | mg | 100 | μg |
| Pycnogenol | 50 | mg | 50 | μg |

### Example 8 Compositions with Branched Chain Amino Acids (BCAA's)

### Composition 11:

| | Recommended daily | Composition |
|---|---|---|
| Compound | intake | (Example) |
| BCAA composition | 4000-6000 mg | 600-1000 μg |

### Example 9 Compositions for Improving Skin Elasticity and Slowing Down Skin Aging

### Composition 12:

| | Recommended daily | Composition |
|---|---|---|
| Compound | intake | (Example) |
| L- Glutamine | 1400 mg | 100 μg |

### Example 10 Compositions for Improving Skin Elasticity, Skin Moisture, Connective Tissue, Cartilage Tissue, and for Reducing Wrinkles

### Composition 13:

| | | |
|---|---|---|
| | Recommended daily | Composition |
| Compound | intake | (Example) |

| | | | | |
|---|---|---|---|---|
| Collagen | 2.5 | g | 2500 | μg |
| Acerola extract | 670 | mg | 500 | μg |
| Vitamin B7 (Biotin) | 50 | μg | 15 | μg |
| Vitamin C | 80 | mg | 100 | μg |
| Vitamin E | 2.3 | mg | 2.33 | μg |
| Zinc | 3 | mg | 3 | μg |

### Example 11 Compositions for Improving Skin Elasticity and Skin Moisture

### Composition 14:

| | Recommended daily | Composition |
|---|---|---|
| Compound | intake | (Example) |
| Collagen hydrolysate | 5000 mg | 1500 μg |

### Example 12 Compositions for Slowing Down the Aging Process and Improving Female and Male Libido

### Composition 15:

### Variant 1

| | Recommended daily | Composition |
|---|---|---|
| Compound | intake | (Example) |
| Wild Yams Root extract | 1000 mg | 800 μg (80%) |

### Composition 16:

### Variant 2

| | Recommended daily | Composition |
|---|---|---|
| Compound | intake | (Example) |
| DHEA | 25 mg for women | 1000 μg |
| (Dehydroepiandrosteron) | 50 mg for men | |

Further additives that may be combined with the above Wild Yams Root extract or DHEA include the following:

| | Recommended daily | Composition |
|---|---|---|
| Compound | intake | (Example) |
| Vitamin E | 12 mg | 100 μg |
| Zinc | 12 mg | 100 μg |

### Example 13 Compositions for Reducing Wrinkles

### Composition 17:

| | Recommended daily | Composition |
|---|---|---|
| Compound | intake | (Example) |
| Hyaluronic acid | 200 mg | 600 μg |

### Example 14 Compositions for Regeneration of Cells in General as Well as for Regeneration of Neurons, and Cerebral Cells

### Composition 18:

| | | |
|---|---|---|
| | Recommended daily | Composition |
| Compound | intake | (Example) |

| | | | | |
|---|---|---|---|---|
| Vitamin B 12 | 500 | μg | 100 | mg |
| Phosphonoserine | 40 | mg | 10 | mg |
| Glutamine | 60 | mg | 15 | mg |

### Example 15 Compositions for Improving the Elasticity and Firmness of the Skin, Skin Rejuvenation, Reduction of Stretch Marks and Skin Pigmentation

### Composition 19:

| | Recommended daily | Composition |
|---|---|---|
| Compound | intake | (Example) |
| Organic silica | 0.5% | 0.5% |
| DMAE | 2.5% | 2.5% |
| Vitamin C | 100 mg | 100 μg |

Liquids with Extracts

### Example 16 Compositions for Improving Memory and Concentration

### Composition 20:

| | Recommended daily | Composition |
|---|---|---|
| Compound | intake | (Example) |
| Ginkgo-Biloba extract | 100 mg | 1000 μg |
| | | (95-100%) |

### Example 17 Antioxidant Compositions

### Composition 21:

| | Recommended daily | Composition |
|---|---|---|
| Compound | intake | (Example) |
| Astaxanthin | 4 mg | 800 μg |
| Vitamin E | 7 mg | 100 μg |

### Example 18 Compositions for Reducing Inflammatory Processes

### Composition 22:

| | Recommended daily | Composition |
|---|---|---|
| Compound | intake | (Example) |
| Boswellia extract | 800-1200 mg | 1000 μg |

### Example 19 Antioxidant Compositions, Anti-Inflammatory Compositions

### Composition 23:

| | Recommended daily | Composition |
|---|---|---|
| Compound | intake | (Example) |
| OPC-grape seed extract | 400 mg | 800 μg |

Further additives that may be combined with the above OPC-grape seed extract include the following:

| | | |
|---|---|---|
| | Recommended daily | Composition |
| Compound | intake | (Example) |

| | | | |
|---|---|---|---|
| Vitamin C | 1000 | mg | 100 μg |
| Vitamin E | 7 | mg | 100 μg |

### Example 20 Antioxidant Compositions and Compositions for Stimulating the Immune System

### Composition 24:

| | Recommended daily | Composition |
|---|---|---|
| Compound | intake | (Example) |
| Pomegranate seed extract | 600-1200 mg | 800 μg |

Further additives that may be combined with the above Pomegranate seed extract include the following:

| | | |
|---|---|---|
| | Recommended daily | Composition |
| Compound | intake | (Example) |

| | | | | |
|---|---|---|---|---|
| Vitamin C | 100 | mg | 100 | μg |
| Zinc | 5 | mg | 5 | μg |
| Selenium | 50 | μg | 0.5 | μg |

### Example 21 Antioxidant Compositions

### Composition 25:

| | Recommended daily | Composition |
|---|---|---|
| Compound | intake | (Example) |
| Acai-berry extract | 4000-6000 mg | 600-1000 μg |

### Example 22 Compositions for Improving Virility, i.e. Male Libido Improving Compositions, Compositions for Maintaining Normal Spermatogenesis and Fertility

### Composition 26:

| | | |
|---|---|---|
| | Recommended daily | Composition |
| Compound | intake | (Example) |

| | | | | |
|---|---|---|---|---|
| Potency Wood extract | 639 | mg | 213 | μg |
| Damiana leaf extract | 639 | mg | 213 | μg |
| Sabal fruits extract | 319.5 | mg | 106 | μg |
| Guarana powder | 127.8 | mg | 43 | μg |

Further additives that may be combined with the above Resveratrol include the following:

| | | |
|---|---|---|
| | Recommended daily | Composition |
| Compound | intake | (Example) |

| | | | | |
|---|---|---|---|---|
| Zinc | 15.3 | mg | 5.1 | μg |
| Selenium | 69 | m | 25 | μg |
| Vitamin B6 | 4.14 | m | 1.4 | μg |

### Example 23 Antioxidant Compositions and Compositions for Protecting from Virus Infections, Such as Influenza and Cold Viruses

### Composition 27:

| | | |
|---|---|---|
| | Recommended daily | Composition |
| Compound | intake | (Example) |

| | | | | |
|---|---|---|---|---|
| Cistus Incanus | 1000 | mg | 800 | μg |
| Vitamin C | 150 | mg | 150 | μg |
| Zinc | 50 | mg | 50 | μg |

### Example 24 Compositions with Creatine

### Composition 28:

| | Recommended daily | Composition |
|---|---|---|
| Compound | intake | (Example) |
| Creatine Monohydrate | 3500 mg | 3000 μg |

### Example 25 Compositions for Improving Concentration

### Composition 29:

| | Recommended daily | Composition | |
|---|---|---|---|
| Compound | intake | (Example) | |
| Caffeine Anhydrous | 270 mg | 4-40 | mcg |
| Green Coffee extract | 200 mg | 100 | mcg |

| | |
|---|---|
| L-Theanine | 100 mg |

### Example 26 Antioxidant Compositions and Compositions for a Functioning Immune System and Metabolism

### Composition 30:

| | | |
|---|---|---|
| | Recommended daily | Composition |
| Compound | intake | (Example) |

| | | | |
|---|---|---|---|
| Acerola extract | 400 | mg | 750 μg (75%) |
| Vitamin C | 70 | mg | 100 μg |

### LIST OF REFERENCE NUMERALS

100 e-cigarette
1 dispensing device
10 dispensing unit
12 composition reservoir
13 reservoir seating
14 air inlet opening
15 cab
16 connector
2 vaporizing unit
20 heater element
22 vaporizing chamber
24 reservoir outlet
26 outer shell
3 flow channel
30 connection socket
4 liquid reservoir
5 counterpart connector
50 sealing
52 end face
6 power supply
7 control circuit arrangement
70 switch
8 mouthpiece
80 mouthpiece outlet

1. A dispensing arrangement (1) for treating a flow of vaporized liquid provided via a flow channel (3) of an e-cigarette (100), comprising
a dispensing unit (10) for adding an additional composition into the flow of vaporized liquid of the e-cigarette (100); and
a composition reservoir (12) for retaining the additional composition,
wherein the dispensing unit (10) is in communication with a composition reservoir (12) and in communication with the flow channel (3), and
wherein the composition reservoir (12) is arranged separately to the flow channel (3), characterized in that
the dispensing arrangement (1) is adapted to temper the vaporized liquid and/or the additional composition to a temperature of below 40° C. or below 35° C. before adding the additional composition into the flow of vaporized liquid.

2. The dispensing arrangement (1) according to claim 1, wherein the mixture of the additional composition and the vaporized liquid has a temperature of below 40° C. or below 35° C.

3. The dispensing arrangement (1) according to claim 1, wherein a cooling device is provided for cooling the additional composition and/or the flow channel (3) such that the additional composition has a temperature of lower than 40° C. or lower than 35° C. and/or the vaporized liquid has a temperature of lower than 40° C. or lower than 35° C.

4. The dispensing arrangement (1) according to claim 1, wherein at least a connector (16) for connecting the dispensing arrangement (1) and the e-cigarette (100) is arranged at a side facing the e-cigarette (100) and/or a mouthpiece (8) or a mouthpiece connection is arranged at a side facing the user.

5. (canceled)

6. The dispensing arrangement (1) according to claim 1,
wherein the composition reservoir (12) comprises a porous solid body coated and/or impregnated and/or soaked with the additional composition, wherein the dispensing unit (10) is provided in the form of passageways in the porous solid body; or
wherein the composition reservoir (12) is a container for receiving the composition in the form of a liquid or powder.

7. The dispensing arrangement (1) according to claim 1, wherein at least one air inlet opening (14) is provided such that a fluidic connection is provided through the composition reservoir (12) from the at least one air inlet opening (14) to the dispensing unit (10).

8. The dispensing arrangement (1) according to claim 1, wherein the composition reservoir (12) contains an additional composition, optionally wherein the additional composition comprises
at least one temperature-sensitive compound, and/or
at least one compound having a poor solubility in the vaporized liquid, and/or
at least one anti-aging compound;
preferably wherein the additional composition comprises at least one temperature-sensitive anti-aging compound.

9. An e-cigarette (100) for the oral intake of a vaporized liquid, comprising a liquid reservoir (4) for retaining a liquid, a flow channel (3) and a vaporizing unit (2) for vaporizing the liquid, characterized in that the e-cigarette (100) further comprises a dispensing arrangement (1) according to claim 1.

10. The e-cigarette (100) according to claim 9, wherein
the dispensing arrangement (1) is connectable with the e-cigarette (100) by means of a counterpart connector; or wherein the dispensing arrangement (1) is integrated in the e-cigarette;
a negative pressure can be applied to the flow channel (3), allowing the addition of the additional composition into the flow of vaporized liquid by a passive intake mechanism due to the negative pressure; and/or
the addition of the additional composition is achieved by a controlled injection of the additional composition into the flow of vaporized liquid.

11.-12. (canceled)

13. A method for treating a flow of vaporized liquid provided by an e-cigarette (100), characterized in that the method comprises the steps of:
providing a liquid to be vaporized;
providing an additional composition;
vaporizing the liquid;
causing a flow of the vaporized liquid, and
adding the additional composition into the flow of vaporized liquid;
wherein the temperature of the vaporized liquid and/or the additional composition is/are tempered to a temperature below 40° C. or below 35° C., or below 30° C. before adding the additional composition into the flow of vaporized liquid; and/or
wherein the mixture of the additional composition and the vaporized liquid has a temperature of below 40° C., or below 35° C., or below 30° C.

14. The method according to claim 13, wherein the additional composition comprises
at least one temperature-sensitive compound, and/or
at least one compound having a poor solubility in the vaporized liquid, and/or
at least one anti-aging compound;
preferably wherein the additional composition comprises at least one temperature-sensitive anti-aging compound.

15. The method according to claim 13, wherein
the additional composition is directly added into the flow of vaporized liquid; and/or
the additional composition is added into an additional air flow which is then mixed with the flow of vaporized liquid.

16. (canceled)

17. The method according to claim 13, wherein
the step of adding the additional composition into the flow of vaporized liquid is provided by applying a negative pressure which causes a passive intake of the additional composition into the flow of vaporized liquid; and/or
the step of adding the additional composition into the flow of vaporized liquid is provided by injecting the additional composition into the flow of vaporized liquid in a controlled fashion.

18. (canceled)

19. Use of a dispensing arrangement (1) according to claim 1, or of an e-cigarette (100) comprising the dispensing arrangement (1), a liquid reservoir (4) for retaining a liquid, a flow channel (3) and a vaporizing unit (2) for vaporizing the liquid, for the oral intake of an additional composition comprising at least one anti-aging compound.

20. The use according to claim 19, wherein the composition reservoir (12) comprises the additional composition comprising at least one anti-aging compound in solid or liquid form;
optionally wherein the additional composition comprising at least one anti-aging compound is present in the form of a vapor, an aerosol, an emulsion or a suspension.

21. The use according to claim 19, wherein the composition reservoir (12) comprises a vaporizable or nebulizable liquid composition comprising a liquid carrier composition and at least one anti-aging compound, wherein said vaporizable liquid composition is present in the form of a solution, emulsion or dispersion.

22. The use of according to claim 19, wherein the composition comprising at least one anti-aging compound is converted to a vapor, an aerosol, an emulsion or a suspension prior to the oral intake at a temperature of below about 40° C., or below about 35° C., or below about 30° C.

23. The use according to claim 19, wherein the liquid carrier composition comprises a mixture of liquid polyalcohols and water, preferably wherein the liquid carrier comprises a mixture of 1,2-propanediol with water, and, optionally, further comprising 1,2,3-propanetriol (glycerol) and/or ethanol; optionally wherein the composition of the first and the second liquid carrier is different from each other.

24. The use according to claim 19, wherein the at least one anti-aging compound is selected from the group consisting of: Acai-berry extract, Acerola extract, BCAA extract, Boswellia extract, Caviar extract, Cistus Incanus, Damiana leaf extract, Ginkgo-Biloba extract, Pomegranate seed extract, Green Coffee extract, OPC-grape seed extract, Prunus Cerasus/bitter cherry extract, Sabal fruits extract, Potency Wood extract, Wild Yams Root extract, Zingiber Officinals/ginger root extract, Butyrospermum Parkii (shea butter), Guarana powder, caffeine, L-theanine, collagen, collagen hydrolysate, creatine monohydrate, hyaluronic acid, L-carnitine, astaxanthin, bioflavonoids, dimethylaminoethanol (DMAE), dehydroepiandrosteron (DHEA), omega-3 fatty acids, docosahexaenic acid (DHA), eicosapentaenic acid (EPA), L-arginine, L-glutamine, L-isoleucine, L-leucine, L-lysine, L-valine, phosphonoserine, Somatotropin (HGH, Human Growth Hormone), Vitamin A (retinol), Vitamin B1 (thiamine), Vitamin B2 (riboflavin), Vitamin B3 (niacin), Vitamin B5 (pantothenic acid), Vitamin B6 (pyridoxine), Vitamin B7 (biotin), Vitamin B9 (folic acid), Vitamin B12 (cobalamin), Vitamin C (ascorbic acid), Vitamin D (ergo-/cholecalciferol), Vitamin E (tocopherol), alpha-tocotrienol, Vitamin K, Coenzyme Q10, organic silica, pycnogenol, resveratrol, magnesium, selenium, zinc, gold particles, and mixtures of any of the foregoing compounds or compositions/extracts.

25. The use according to claim 19, wherein the composition comprising the at least one anti-aging compound contains at least 2, 3, 4, 5, 6, 7, 8, 9, or at least 10 different anti-aging compounds; optionally wherein the composition further comprises a flavorant.
